(19) United States
(12) Patent Application Publication
(10) Pub. No.: US 2021/0369382 A1
(43) Pub. Date: Dec. 2, 2021

(54) Barrier Shield

(71) Applicant: Brian Del Core, Aptos, CA (US)

(72) Inventor: Brian Del Core, Aptos, CA (US)

(21) Appl. No.: 16/886,351

(22) Filed: May 28, 2020

(51) Int. Cl. A61B 90/00; A61B 90/50

(52) U.S. Cl. CPC A61B 90/05; A61B 2217/005; A61B 90/50

(57) ABSTRACT

A shield assembly has a transparent shield element, having an open first end, a second end, and downward curving sides, a mounting plate shaped in the curvature of the shield joined by an upper surface of the mounting plate to the shield element at the second end, a vacuum casing with a first closed end and a second open end wider than the first end joined to a lower surface of the mounting plate within the elongated shield element between the downward curving sides, a vacuum hose connected from a vacuum pump by a quick coupling through the first closed end to inside the vacuum casing, and a medical grade filter covering the second open end of the vacuum casing. Operating the vacuum pump causes air to be drawn under the elongated shield element through the filter into the vacuum casing and through the vacuum hose and the vacuum pump.

### BACKGROUND OF THE INVENTION

### 1. Field of the Invention

The present invention is in the field of medical support equipment and pertains more particularly to methods and apparatus for shielding a service provider from a service recipient.

### 2. Discussion of the State of the Art

Medical support equipment includes personal protection equipment (PPE) like masks, gowns, hair nets, and gloves to be worn by a service provider, such as nurses and doctors providing medical treatment to patients. Medical facilities like dental offices, physician's rooms and surgery rooms necessitate close quarters between a service provider and a receiver of services.

One challenge with treating patients in close quarters is the prospect that viruses may be airborne and may travel through air and infect patients and or the service provider without adequate protection. This problem has more recently been experienced in association with a virus pandemic associated with the Covid-19 virus occurring at the time of this writing. A significant number of medical personnel have been infected in this pandemic.

Therefore, what is clearly needed is a much-improved personal barrier shield for protecting parties from exposure to a virus.

### BRIEF SUMMARY OF THE INVENTION

In one embodiment of the invention a shield assembly is provided, comprising an elongated shield element of transparent material, having an open first end, a second end, and downward curving sides, a mounting plate shaped in the curvature of the shield joined by an upper surface of the mounting plate to the shield element at the second end, a vacuum casing with a first closed end and a second open end wider than the first end joined to a lower surface of the mounting plate within the elongated shield element between the downward curving sides, a vacuum hose connected from a vacuum pump by a quick coupling through the first closed end to inside the vacuum casing, and a medical grade filter covering the second open end of the vacuum casing. Operating the vacuum pump causes air to be drawn under the elongated shield element through the filter into the vacuum casing and through the vacuum hose and the vacuum pump.

In one embodiment the shield assembly further comprises a screen in the shape and size of the open end of the vacuum casing, with the filter covering the open end held in place by the screen in a manner that the screen is removable to replace the filter. Also, in one embodiment the shield assembly further comprises an articulated arm assembly joined by a first arm at one end to the mounting plate at the second end of the shield element, and by a second arm to a support. In one embodiment the support is a carriage with a wheeled base and an upwardly extending post having a collar translatable on the post with the second arm joined to and extending from the collar, such that the articulated arm assembly is adapted to be positioned at different heights, also positioning the shield assembly at different heights. And in one embodiment the shield assembly further comprises a third arm joined by a first pivot joint at one end to the first arm and by a second pivot joint at the opposite end to the second arm, such that the arms may be adjusted to adjust the angle of the shield element with horizontal.

In one embodiment the first arm is a telescoping arm enabling the shield element may be translated toward and away from the support. Also, in one embodiment the support is a wall of a room in which the shield assembly is deployed. Also, in one embodiment the vacuum pump has an exhaust port, and further comprising conduit from the exhaust port passing through a wall to outside a building where the shield assembly is deployed.

In another aspect of the invention a method for containing and managing effluent from a patient is provided, comprising positioning an elongated shield element of transparent material, having an open first end, a second end, and downward curving sides, with a mounting plate shaped in the curvature of the shield joined by an upper surface of the mounting plate to the shield element at the second end, and a vacuum casing with a first closed end and a second open end wider than the first end joined to a lower surface of the mounting plate within the elongated shield element between the downward curving sides, over the head of the patient with the patient facing upward, and drawing air from under the elongated shield element through a vacuum hose connected from a vacuum pump by a quick coupling through the first closed end to inside the vacuum casing.

In one embodiment the method further comprises placing a medical grade filter over the second open end of the vacuum casing. In one embodiment the method further comprises placing a screen in the shape and size of the open end of the vacuum casing, the filter covering the open end held in place by the screen in a manner that the screen is removable to replace the filter. In one embodiment the method further comprises supporting the elongated shield element by an articulated arm assembly joined by a first arm at one end to the mounting plate at the second end of the shield element, and by a second arm to a support.

In one embodiment the method further comprises attaching the articulated arm assembly to a carriage with a wheeled base and an upwardly extending post having a collar translatable on the post, with the second arm joined to and extending from the collar, such that the articulated arm assembly is adapted to be positioned at different heights, also positioning the shield assembly at different heights. In one embodiment the method further comprises placing a third arm joined by a first pivot joint at one end to the first arm and by a second pivot joint at the opposite end to the second arm, such that the arms may be adjusted to adjust the angle of the shield element with horizontal. In one embodiment the first arm is a telescoping arm enabling the shield element may be translated toward and away from the support.

In one embodiment the method further comprises connecting the articulated arm assembly to a wall of a room in which the shield assembly is deployed, as support. And in one embodiment the method further comprises connecting a conduit from an exhaust port of the vacuum pump passing through a wall to outside a building within which the shield assembly is deployed.

### BRIEF DESCRIPTION OF THE SEVERAL VIEWS OF THE DRAWINGS

FIG. 1 is an overhead plan view of a barrier shield assembly according to an embodiment of the present invention.

FIG. 2 is a right-side view of the barrier shield assembly of FIG. 1.

FIG. 3 is a front-end view of the barrier shield assembly of FIG. 1.

FIG. 4 is an exploded view of a vacuum housing assembly of the barrier shield assembly of FIG. 1

FIG. 5 is an architectural view of the barrier shield assembly of FIG. 1. connected to system components according to an embodiment of the present invention.

FIG. 6 is an enlarged partial view of the barrier shield assembly of FIG. 1. connected to system components of FIG. 5.

### DETAILED DESCRIPTION OF THE INVENTION

In various embodiments described in enabling detail below the inventor teaches a unique system for preventing airborne viruses from transmission between persons in close quarters, such as in procedures in the medical and dental professions. The present invention is described using the following examples, which may describe more than one relevant embodiment falling within the scope of the invention.

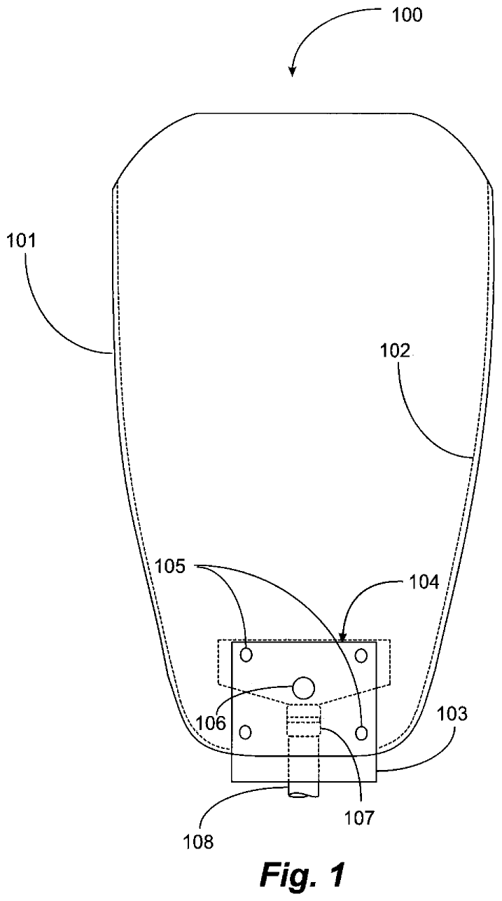

FIG. 1 is an overhead view of a barrier shield assembly 100 according to an embodiment of the present invention. Barrier shield assembly 100 includes a barrier shield 101. Barrier shield 101 may in one embodiment be formed from an acrylic sheet that is scratch resistant and optically transparent. Barrier shield 101 has a length and a width and a material thickness. Barrier shield 101 may be formed in one process by heat form bending or similar process to urge the long edges of the sheet downward (in this view) on either side of center to form a half tunnel with open ends.

In one implementation barrier shield 101 is an optically clear material such as acrylic or safety glass that is resistant to deforming, scratching, or marring under normal use. Barrier shield 101 is adapted in different embodiments as a safety barrier between a medical worker or other service provider and a patient to reduce the level of airborne contaminants, including viruses, that may cross the small airspace between the parties to a medical procedure where a practitioner must be in close contact with the patient.

Barrier shield 101 in one embodiment is impact resistant in accordance with existing safety standards. Barrier shield 101 is of a material that may safely be cleaned using solutions made for acrylic or safety glass. In one embodiment, food grade materials may be used that can be sterilized by heat sterilization methods without showing degradation of surface translucency or surface uniformity. The barrier shield may be specifically crafted for application requirements such as surgical grade shields for medical safety applications to less stringent versions of the shield for applications not requiring complete sanitation.

Barrier shield assembly 100 in one embodiment includes a mounting plate 103 upon which barrier shield 101 is mounted using conventional hardware, such as fasteners 105. Mounting plate 103 provides a stable mounting point for mounting barrier shield assembly 100 to a mechanically operated or a robotically controlled apparatus having an articulating arm for positioning barrier shield 101 for use in a medical application or provider service. A knob 106 is connected to a stop screw to tighten the barrier shield assembly 100 to a desired rotational angle of presentation (horizontally) in mounted position on an articulating arm of a positioning apparatus. This is described in more detail below.

In one embodiment, barrier shield assembly 100 includes a vacuum head assembly 104. Vacuum head assembly 104 may have a molded casing that holds a replaceable air filter and a cover screen. The casing may be molded from a food grade plastic and may be mounted to and removed from barrier shield 101 and cleaned and sterilized and re installed for use.

Vacuum head assembly 104 is adapted to be quick connected to a vacuum hose 108 via a quick connect attachment 107. Vacuum hose 108 may be routed to a vacuum pump with an exhaust outlet or a high-volume evacuation (HVE) system such as implemented in dental offices. The components attached to barrier shield 101 to form assembly 100 may be removed and isolated for sterilization, then reassembled for subsequent use. Moreover, replacement shield assemblies like assembly 100 may be available as replacement assemblies reducing any downtime during sterilization procedures.

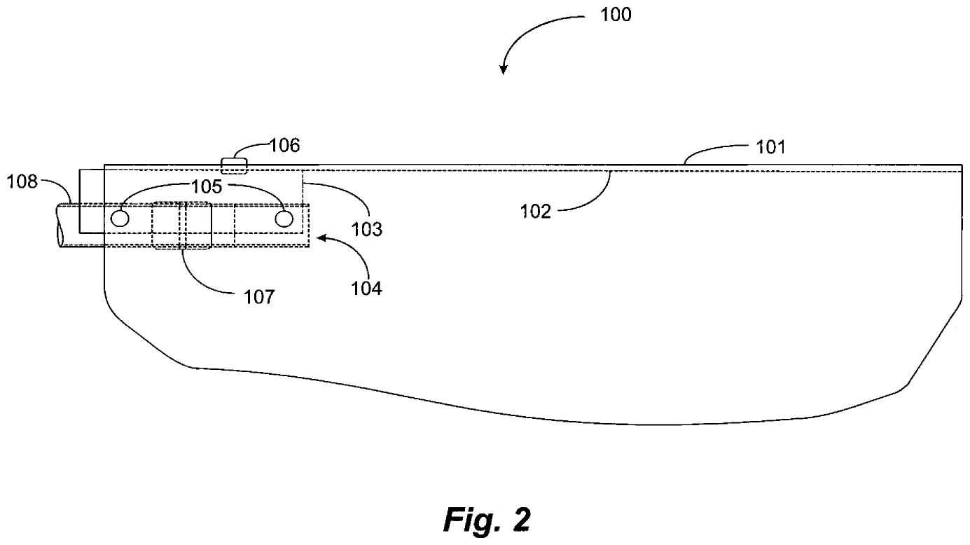

FIG. 2 is a right-side view of barrier shield assembly 100 of FIG. 1. Barrier shield 101 may be formed with arcuate sides extending out and downward to form an inverted half tunnel structure that inhibits cross airflow and creates a funnel structure for air drawn into vacuum head assembly 104. The form of the sides of the shield may be annular or arcing downward or angular or angled downward by multiple bends having the progressive effect of forming the tunnel structure.

The downward extending sides of barrier shield 101 measured from the back surface of the shield to the downward extended edges may have a greater height dimension further distant away from vacuum head assembly. Thickness of barrier shield 101 determines underside surface 102 of the shield and inside surfaces of the shield wall.

In one embodiment, vacuum head assembly 104 is removably attached to barrier shield 101 on the underside surface of mounting plate 103, which has a curved aspect matching the curvature of the shield. In one embodiment vacuum head assembly 104 is fixedly aligned longitudinally with barrier shield 101. Knob 106 may connect to a rotational mounting component (not illustrated) of an articulating arm, shown further below. Barrier shield assembly 100 is positioned in between a medical or service provider and a patient.

One example of the above use case might be shielding a dentist from the breath of a dental patient undergoing a dental procedure. The shape of the barrier shield keeps the exhalations of the patient beneath the barrier. Negative air flow underneath the barrier through vacuum head assembly 104 draws in the patient's exhalation and ambient air and filters that air while preventing the potentially infected air from rising above the barrier shield where it might infect a dentist or an assistant.

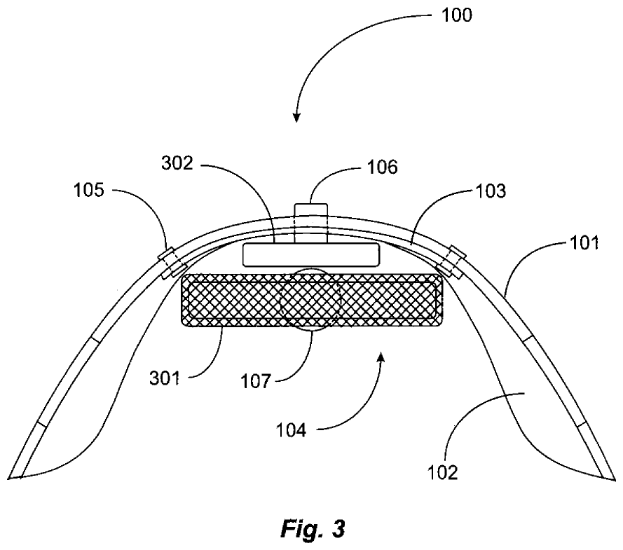

FIG. 3 is a front-end view of barrier shield assembly 100 of FIG. 1. Barrier shield 100 is viewed from the perspective of a patient in this view where the patient may be lying flat on a table or bed. Vacuum head assembly 104 faces the patient and creates a negative air flow from the patient into the assembly and out to an exhaust as described further above. Vacuum head assembly 104 may be removably mounted to barrier shield 101, perhaps at the edges of mounting plate 103, in a position substantially aligned with the longitudinal axis of barrier shield 101.

Barrier shield assembly 100 includes a rotational mounting disc 302. Mounting disc 302 may provide a rotationally adjustable mounting position to an articulating arm segment of a positional apparatus used to position the shield between provider and patient. It may be noted herein that horizontal rotational adjustment may be made from the top surface of barrier shield 101 via manipulation of knob 106.

In one embodiment, vacuum head assembly 104 is fixed to the barrier shield and rotates with the shield. In another embodiment, vacuum head assembly 104 is mounted to the articulating arm segment rotational component 302. In the latter embodiment, vacuum head assembly 104 stays with the positioning apparatus when a shield is removed for re sterilization between uses. In one embodiment, vacuum head assembly 104 is mounted using the same hardware for mounting plate 103 to inside surface 102 of barrier shield 101.

Vacuum head assembly 104 includes a removable protective screen 301 that may be fabricated of a medical grade stainless steel or plastic. Protective screen 301 may be adapted with a frame component to couple to the wide vacuum end of the assembly as an elongated cap. A replaceable filter material or filter pad (not illustrated) may be installed within the vacuum end of vacuum head assembly 104 for filtering microbes or virus particles and droplets from the vacuumed air including the patient's exhalations before exhausting the air at a point safely away from the patient and medical service provider.

In one embodiment, vacuum hose 108 may be routed through a wall supporting the positioning apparatus for positioning barrier shield assembly 100 for use. In this embodiment, an HVE or vacuum pump exhaust may be routed further to the outside of a building. Contaminated filters may be removed, discarded, and replaced. Protective screen 302 may be sterilized with the other components of the shield assembly.

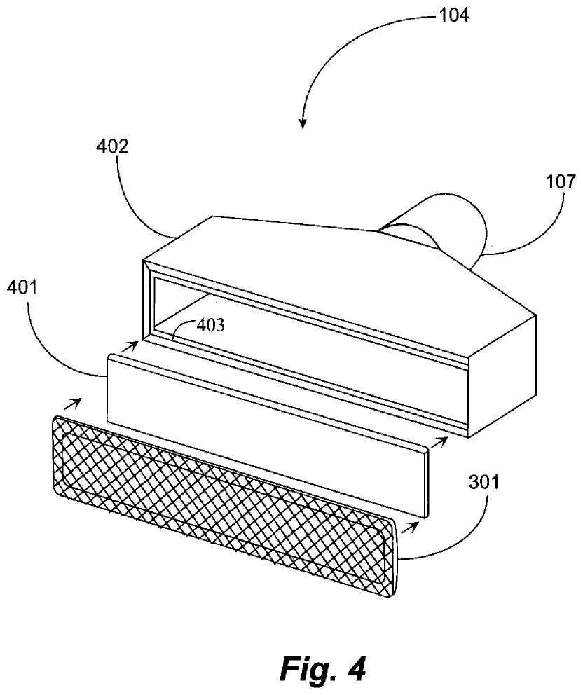

FIG. 4 is an exploded view of vacuum head assembly 104 of barrier shield assembly 100 of FIG. 1. Vacuum shield assembly 104 comprises a body 402 and quick connector 107 for attachment of the vacuum hose. Vacuum head 402 may include a shelf structure machined or otherwise formed around the inner edge of the wide opening for seating a filter 401 for filtering the vacuumed ambient air including a patient's exhalations.

Vacuum filter 401 may be an N-95 grade filter for medical use. Other filter materials capable of trapping airborne particles from a patient's exhalations may be substituted for N-95 mask material without departing from the spirit and scope of the present invention. In this embodiment, filter 401 is placed into vacuum head 402 against shelf 403. Protective screen 301 may then be placed over the end securing the filter in place against the shelf. In some embodiments the filter may be deeper and in the shape of the inside of the body 402.

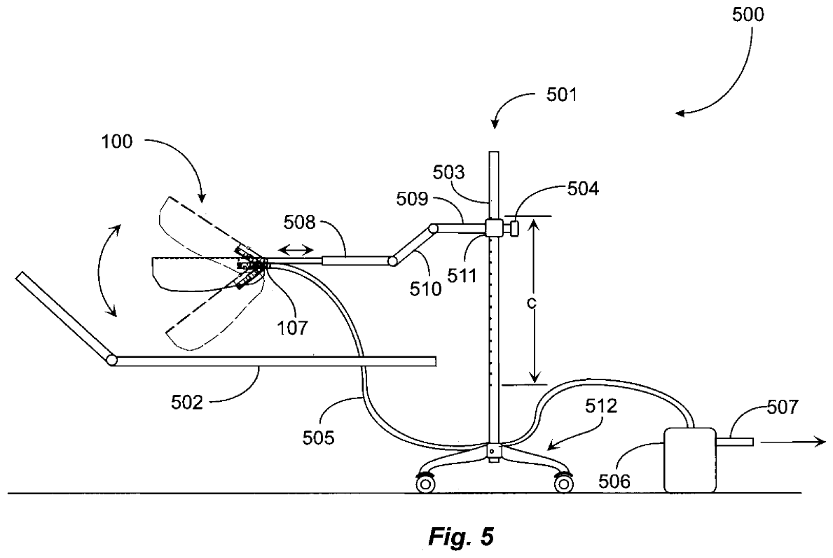

FIG. 5 is an architectural view of the barrier shield assembly 100 of FIG. 1. connected to system components 500 according to an embodiment of the present invention. Components 500 include a positioning apparatus 501 with an articulating arm structure that may be adjustable to mechanically position barrier shield 100 between a patient and a medical service provider, for example. In this embodiment a patient (not illustrated) may lie on patient bed 502 for a medical procedure. Positioning apparatus 501 may include a vertical post 503 connected to a stable wheelbase 512, the wheelbase having at least three wheels including mechanical wheel locks or brakes that may be foot operable.

Positioning apparatus 501 further includes a collar 511 adapted, in this embodiment, to fit slidably over post 503. Collar 511 includes a spring tensioned pin clamp having a pull knob 504 and a pin (not illustrated) that fits into one of a plurality openings provided through the sidewall of post 503 in a straight vertical hole pattern referenced herein by dimension C depicting the range of openings. Collar 511 is depicted not engaged and rotated out of engagement alignment as may be the case during height adjustment of the collar along the post. Collar 511 supports an articulating arm structure on the side of the collar opposite from pin clamp 504.

The articulating arm structure on positional apparatus 501 includes a first arm segment 509, a second arm segment 510, and a third arm segment 508. Arm segment 509 is seated within collar 511. Arm segment 510 is a middle arm segment having rotable connections to arm segment 510 and arm segment 508. Arm segment 508 includes an extendible and retractable telescopic rod portion for extending the barrier shield assembly 100 further forward or retracting the barrier shield assembly back toward the positioning apparatus 501.

In one embodiment, the articulating arm structure comprising arm segments 508, 509, and 510 may include individual tightening elements (not illustrated) at each rotational coupling between the segments so that they may be articulated and then tightened into a fixed extended position to present barrier shield assembly 100 in a desired position for use. In one embodiment, collar 511 may be adapted to fit over post 503 and navigate a vertical track attached to the post.

In one embodiment, positional apparatus 501 may be a robotic track system vertically mounted on a wall that may be automated by a linear actuator controlled by a remote-control device to raise and lower barrier shield assembly 100. In another embodiment, the articulating arm may be a wire flex conduit that a user may manipulate to position the shield at a desired angle and height relative to a patient. A cover or drape, such as plastic sheeting, may be used to cover positional apparatus 501 and barrier shield 100 when not in use to prevent dust or other contaminants from contact with the sterilized components.

A medical service provider may physically roll positioning apparatus 501 into a desired position and may physically adjust the height of and extension metrics for barrier shield assembly 100. Barrier shield assembly 100 may be pivoted vertically up or down as indicated by the broken image boundaries and the directional arrow depicting upward and downward tilt direction. Positioning apparatus 501 maybe be a free standing system or a roll able cart or tray system, or a wall-mounted system using a linear track, or a post mounted system clamped to a table, cart, or other structure without departing from the spirit and scope of the invention.

Vacuum hose 505 (analogous to vacuum hose 108, FIG. 1) may be connected to a vacuum source like a vacuum pump 506 having an exhaust outlet 507 for expelling the vacuumed and filtered air. In one embodiment exhaust hose 507 is routed to outside of the building for dilution in open air. Barrier shield 101 may be approximately two or two- and one-half times as long as a patient's head and perhaps twice as wide as the patient's head. The height of the curve formed by barrier shield assembly 100 may be just deeper than a patient's head from the back of the patient's skull to the tip of the patient's nose. Other shapes and sizes may be provided for different applications without departing from the spirit and scope of the present invention.

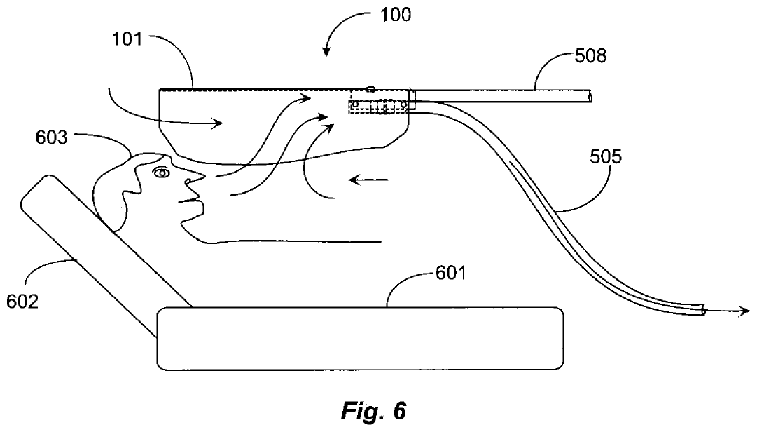

FIG. 6 is an enlarged view of barrier shield assembly 100 of FIG. 1. in position over a patient 603. Barrier shield assembly 100 is in fixed position over patient 603 on a hospital bed 601 with an adjustable back rest 602. Ambient air including exhalations of the patient are drawn into barrier shield 101 according to the direction of the multiple arrows depicting air flow. Exhalations from patient 603 are directed into the inverted tunnel form of the shield structure and channeled toward the vacuum head where the air enters through a screen and filter and is pumped out through vacuum hose 505. As negative air flow continues, ambient air above the shield structure may be drawn down and into the channel of the structure as well as ambient air from behind the vacuum head assembly.

The vacuum head assembly 104 would ideally be a relatively low-profile design and located on the area furthest from the operating field and positioned near the inner surface 102 of the inside of the shield. In one embodiment, additional air flow elements may be added to barrier shield assembly 100 to increase negative airflow pressure perhaps along the inner periphery further inhibiting cross flow or potential escape of exhaled particles past the edges of the shield. Additional airflow elements may also be installed on the positional apparatus 501 such as on a separate or on the same articulating arm structure.

Attachments to the HVE system or a separate filtration unit may be connected and disconnected using quick-release coupling hardware that may be amenable to cleansing and to sterilization practices. Filter 401 (FIG. 4) may be a single use disposable filter to protect the HVE or filtration system from contamination. Air removed from the field of use would not be recirculated, but rather vented away from the immediate operating environment. An exterior vent to the outside of the building would be preferred in some embodiments.

It will be apparent to one with skill in that art that the present invention may be enabled using some or all the elements described without departing from the spirit and scope of the present invention. The present invention is described in different embodiments, each an implementation of the invention not to be construed to be limiting. The invention is limited only by the breadth of the claims below.

I claim:

1. A shield assembly, comprising:
an elongated shield element of transparent material, having an open first end, a second end, and downward curving sides;
a mounting plate shaped in the curvature of the shield joined by an upper surface of the mounting plate to the shield element at the second end;
a vacuum casing with a first closed end and a second open end wider than the first end joined to a lower surface of the mounting plate within the elongated shield element between the downward curving sides;
a vacuum hose connected from a vacuum pump by a quick coupling through the first closed end to inside the vacuum casing; and
a medical grade filter covering the second open end of the vacuum casing;
wherein operating the vacuum pump causes air to be drawn under the elongated shield element through the filter into the vacuum casing and through the vacuum hose and the vacuum pump.

2. The shield assembly of claim 1 further comprising a screen in the shape and size of the open end of the vacuum casing, with the filter covering the open end held in place by the screen in a manner that the screen is removable to replace the filter.

3. The shield assembly of claim 1 further comprising an articulated arm assembly joined by a first arm at one end to the mounting plate at the second end of the shield element, and by a second arm to a support.

4. The shield assembly of claim 3 wherein the support is a carriage with a wheeled base and an upwardly extending post having a collar translatable on the post with the second arm joined to and extending from the collar, such that the articulated arm assembly is adapted to be positioned at different heights, also positioning the shield assembly at different heights.

5. The shield assembly of claim 3 further comprising a third arm joined by a first pivot joint at one end to the first arm and by a second pivot joint at the opposite end to the second arm, such that the arms may be adjusted to adjust the angle of the shield element with horizontal.

6. The shield assembly of claim 5 wherein the first arm is a telescoping arm enabling the shield element may be translated toward and away from the support.

7. The shield assembly of claim 3 wherein the support is a wall of a room in which the shield assembly is deployed.

8. The shield assembly of claim 1 wherein the vacuum pump has an exhaust port, and further comprising conduit from the exhaust port passing through a wall to outside a building where the shield assembly is deployed.

9. A method for containing and managing effluent from a patient, comprising:
positioning an elongated shield element of transparent material, having an open first end, a second end, and downward curving sides, with a mounting plate shaped in the curvature of the shield joined by an upper surface of the mounting plate to the shield element at the second end, and a vacuum casing with a first closed end and a second open end wider than the first end joined to a lower surface of the mounting plate within the elongated shield element between the downward curving sides, over the head of the patient with the patient facing upward; and
drawing air from under the elongated shield element through a vacuum hose connected from a vacuum pump by a quick coupling through the first closed end to inside the vacuum casing.

10. The method of claim 9 further comprising placing a medical grade filter over the second open end of the vacuum casing.

11. The method of claim 9 further comprising placing a screen in the shape and size of the open end of the vacuum casing, the filter covering the open end held in place by the screen in a manner that the screen is removable to replace the filter.

12. The method of claim 9 further comprising supporting the elongated shield element by an articulated arm assembly joined by a first arm at one end to the mounting plate at the second end of the shield element, and by a second arm to a support.

13. The method of claim 12 further comprising attaching the articulated arm assembly to a carriage with a wheeled base and an upwardly extending post having a collar translatable on the post, with the second arm joined to and extending from the collar, such that the articulated arm assembly is adapted to be positioned at different heights, also positioning the shield assembly at different heights.

14. The method of claim 12 further comprising placing a third arm joined by a first pivot joint at one end to the first arm and by a second pivot joint at the opposite end to the second arm, such that the arms may be adjusted to adjust the angle of the shield element with horizontal.

15. The method of claim 13 wherein the first arm is a telescoping arm enabling the shield element may be translated toward and away from the support.

16. The method of claim 12 further comprising connecting the articulated arm assembly to a wall of a room in which the shield assembly is deployed, as support.

17. The method of claim 9 further comprising connecting a conduit from an exhaust port of the vacuum pump passing through a wall to outside a building within which the shield assembly is deployed.
